(19) United States
(12) Patent Application Publication
(10) Pub. No.: US 2022/0151575 A1
(43) Pub. Date: May 19, 2022

(54) CUSHIONING AND PROTECTIVE UTILITY ARTICLE FOR A WIRELESS PORTABLE X-RAY DETECTOR

(71) Applicant: Ashenafi Bereded, Triangle, VA (US)

(72) Inventor: Ashenafi Bereded, Triangle, VA (US)

(21) Appl. No.: 17/524,320

(22) Filed: Nov. 11, 2021

Related U.S. Application Data
(60) Provisional application No. 63/115,219, filed on Nov. 18, 2020.

(51) Int. Cl. A61B 6/10; A61B 6/00

(52) U.S. Cl. CPC A61B 6/102; A61B 6/4405

(57) ABSTRACT

A cushioning article for a wireless portable X-ray detector part of a mobile X-ray machine. The cushioning article can cover the wireless portable X-ray detector. The cushioning article can be made from a radiolucent sponge covered with an antimicrobial and waterproof covering.

### CROSS-REFERENCE TO RELATED APPLICATIONS

This application claims priority from the U.S. provisional patent application Ser. No. 63/115,219, filed on Nov. 18, 2020, which is incorporated herein by reference in its entirety.

### FIELD OF INVENTION

The present invention relates to a cushioning article for a wireless portable X-ray detector, and more particularly, the present invention relates to cushioning and protective utility article for the wireless portable X-ray detector of portable X-ray machine.

### BACKGROUND

X-ray is one of the most commonly prescribed imaging modalities for diagnosing a medical condition. Perhaps most of the patients visiting a hospital get an X-ray examination at least once during their visit. Patients in emergency conditions are admitted to an emergency facility in a hospital, wherein X-rays are the primary source of imaging the damage to the patient because it is faster, economic, and easier to perform. The admitted patients in a hospital generally lie in beds and are transported to different departments in a wheelchair. For example, to perform an X-ray examination or other diagnostic test, the patient is taken in a wheelchair to the diagnostic labs or departments. Sometimes, the patient has to walk to the diagnostic labs. Many patients due to their medical condition often find it difficult to stand and sit in the wheelchair or stand for the tests. Thus, in the related medical field, research is mainly focused around bringing the maximum number of diagnostic technologies and methods to the patient rather than the patient going to a lab.

Mobile X-ray machines are such a portable technology in which the X-ray machine is brought to the patient's room and the X-ray can be taken while the patient remains in the bed. Most of the bedside X-ray examinations are chest x-rays. Commonly, these bedside chest X-rays are performed in the following manner. After receiving the examination order from the doctor, the radiology technologist will take the mobile X-ray machine to the patient's room. After the introduction and proper identification of the patient, the radiology technologist prepares the equipment for the examination. In order to image the patient's chest, the technologist uses a combination of the mobile machine, X-ray tubes, and the wireless portable X-ray detector. To place the detector, the patient can be positioned in a semi-upright or lying (supine) position. The radiology technologists then can place the detector plate under or behind the patient's back. At this stage of the procedure, the radiology technologists generally warn the patient that they are about to place a hardboard and it's going to be uncomfortable and might cause pain. Perhaps, the detector is very uncomfortable and even painful for the patients because of the hard and rigid material. Regardless of the warning, some patients may move or turn to get relief from the pressure the hardboard puts on their skin. This movement of the patient may affect the quality of the image produced which in turn leads to misdiagnosis or may cause additional X-ray exposure because of which the examination has to be repeated. In addition, due to the firmness of the detector and the mechanism of placing the detector can cause pressure, shear, and friction injury. The level of discomfort and occurrence of pressure injury is much worse for the elderly and chronically sick patients because they have prominent kyphotic spinal curvature on their back, prominent bony structure, and fragile skin. Currently, to remedy this problem, some radiology technologists use a pillow or a blanket to make the patient comfortable and to avoid related problems during the procedure. But due to lack of uniformity in thickness and radiolucency, which causes an artifact that can degrade image quality, and as a result, the technician might need to repeat the procedure and expose the patient to unnecessary radiation.

Thus, a desire is there for an article that can make the X-ray capturing procedure for mobile X-ray machines comfortable and non-painful.

### SUMMARY OF THE INVENTION

The following presents a simplified summary of one or more embodiments of the present invention in order to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a cushioning article that can cover a wireless portable X-ray detector.

It is another object of the present invention that the cushioning article can protect the detector from physical impacts and environmental contaminants.

It is still another object of the present invention that the cushioning article makes the procedure comfortable for the patients.

It is yet another object of the present invention that the cushioning article may not interfere with X-rays.

It is a further object of the present invention that the cushioning article can be quickly and easily covered to the detector and uncovered.

In one aspect, disclosed is a cushioning article for wireless portable X-ray detectors, wherein the cushioning article can cover the wireless portable X-ray detector. The cushioning article can be made from a radiolucent sponge covered with an antimicrobial and waterproof covering.

These and other objects and advantages of the embodiments herein and the summary will become readily apparent from the following detailed description taken in conjunction with the accompanying drawings.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 shows a rear view of the cushioning article covering a wireless portable X-ray detector, according to an exemplary embodiment of the present invention.

FIG. 2 is a front view of the cushioning article, according to an exemplary embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

Disclosed is a utility article that can be used to cover a wireless portable X-ray detector for cushioning and protection. The utility article can be a cushioning article that makes the X-ray examination comfortable for the patients by providing a degree of cushioning against the hard surface of the detector. The disclosed cushioning article can also protect the detector from external bumps and shocks. Also, the waterproof layer can prevent any harm from dust and accidental spills. The detector when in use can strike to bed or accidentally fall from hands. The disclosed cushioning article can absorb the force from such shock preventing or minimizing the possible damages. The cushioning can also protect the patient from any injury due to the hard surface of the detector.

In one exemplary embodiment, the disclosed cushioning article can be made from a radiolucent sponge that does not cause any interference with X-rays and the imaging may not be affected. In one case, a 1-2 inches thick radiolucent sponge can be used in the cushioning article. The cushioning article can have an outer layer, preferably of a slippery material that may allow the detector covered with the disclosed cushioning article to slide in and out from below the patient easily. For example, the outer layer of the cushioning article can be made from materials with a low coefficient of friction, such as polytetrafluoroethylene or nylon. Preferably, the outer layer can also be dustproof and waterproof to protect the detector from environmental contaminants and accidental spills.

The size of the cushioning article can be proportional to the size of the detection. For example, generally, the detector is manufactured in two standard sizes 14*17 inches and 10*12 inches. The disclosed cushioning article can be manufactured for the standard sizes of the detector.

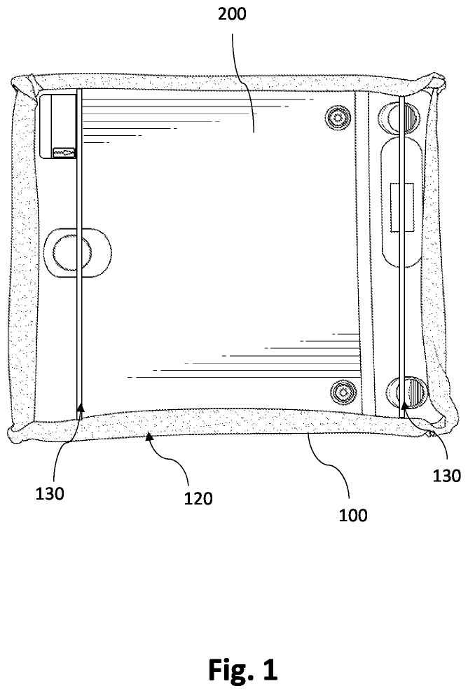

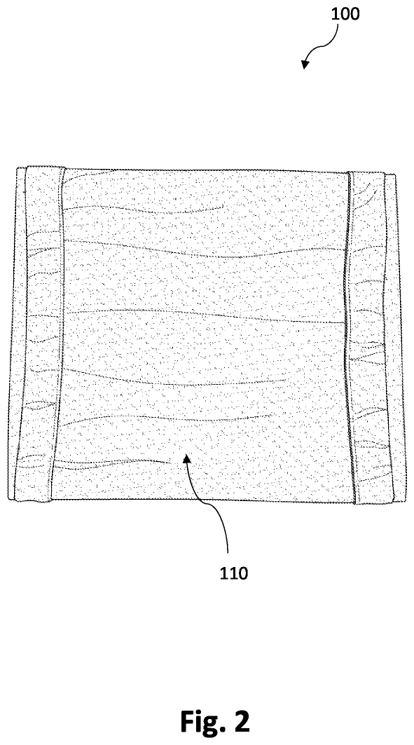

Referring to FIG. 1 which shows the disclosed cushioning article 100 covering a detector 200. The cushioning article 100 can have a planar member 110 (shown in FIG. 2) that can be at least made from the radiolucent sponge. Along a periphery of the planar member 110 can be a continuous skirt 120 that can wrap around the sidewalls of the detector 200. The cushioning article can also include straps 130 to secure the cushioning article to the detector. It is understood that any fastener other than the straps is within the scope of the present invention. In one case, the straps can have hook and loop fasteners for engaging the two straps.

In one implementation of the cushioning article, the cushioning article can be rectangular and 1-2 inches thick. The cushioning article can be put on the detector in a number of ways. In one case, the straps can be provided at four corners of the rectangular cushioning article. The four straps can optionally be elastic and may also include a hook and loop fastener. The straps can be coupled end to end with each other for securing the cushioning article. In another case, the skirt of the cushioning articles can be rigid that can snap-fit to the sidewall corners of the detector. Still, in another case, straps can be provided on two opposite sides of the skirt. The straps can wrap over the detector and be secured by fasteners, such as the hook and loop fastener.

In one implementation of a method for cushioning a wireless portable X-ray detector, the method includes the steps of putting on the cushioning article over the front side of the detector, such as the cushioning article can be between the patient and the detector, the detector placed on a bed over which the patient is lying. After taking the X-ray, the cushion can be removed from the detector, cleaned, and stored for next use. The cushioning article can not only make the bedside (portable) X-ray procedure a comfortable and pain-free experience, but it can also protect any damage to the detector in case of accidental dropping.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A method for cushioning a detector, the detector is a wireless portable X-ray detector of a mobile X-ray machine, the method comprising the steps of:
providing a cushioning article, the cushioning article comprises:
a planar member of a size proportional to a size of the detector,
a skirt extends from a periphery of the planar member, wherein the skirt is configured to fasten to sidewalls of the detector, and
one or more fastening members configured to secure the cushioning article to the detector;
putting on the cushioning article over the detector; and
engaging the one or more fastening members.

2. The method according to claim 1, wherein the planar member is made from a radiolucent sponge.

3. The method according to claim 2, wherein the planar member further comprises an outer layer of waterproof material.

4. The method according to claim 1, wherein the one or more fastening members comprises four straps coupled to four corners of the skirt.

5. The method according to claim 1, wherein the one or more fastening members comprises a plurality of straps coupled to two opposite sides of the skirt.

6. A method for performing X-ray examination using a mobile X-ray machine, the mobile X-ray machine has a detector, the detector is a wireless portable X-ray detector, the method comprising the steps of:
providing a cushioning article, the cushioning article comprises:
a planar member of a size proportional to a size of the detector, and
a skirt extends from a periphery of the planar member, wherein the skirt is configured to fasten to sidewalls of the detector;
putting on the cushioning article over the detector; and
placing the detector below a patient, such as the planar member is between the patient and the detector.

7. The method according to claim 6, wherein the planar member is made from a radiolucent sponge.

8. The method according to claim 7, wherein the planar member further comprises an outer layer of waterproof material.

9. The method according to claim 6, wherein the cushioning article further comprises four straps coupled to four corners of the skirt, the method further comprises the steps of:
engaging the four straps to secure the cushioning article over the detector.

10. The method according to claim 6, wherein the cushioning article further comprises a plurality of straps coupled to two opposite sides of the skirt, the method further comprises the steps of:
engaging the plurality of straps to secure the cushioning article over the detector.

11. A cushioning article for a detector, the detector is a wireless portable X-ray detector of a mobile X-ray machine, the cushioning article comprises:
a planar member of a size proportional to a size of the detector; and
a skirt extends from a periphery of the planar member, wherein the skirt is configured to fasten to sidewalls of the detector.

12. The cushioning article according to claim 11, wherein the skirt is configured to snap-fit over corners of the sidewalls of the detector.

13. The cushioning article according to claim 11, wherein the planar member is made from a radiolucent sponge.

14. The cushioning article according to claim 12, wherein the planar member further comprises an outer waterproof layer.
